(19) United States
(12) Patent Application Publication
(10) Pub. No.: US 2024/0091131 A1
(43) Pub. Date: Mar. 21, 2024

(54) Oral Care Compositions and Methods of Use

(71) Applicant: Colgate-Palmolive Company, New York, NY (US)

(72) Inventors: Payal ARORA, Lebanon, NJ (US); Shashank POTNIS, North Brunswick, NJ (US); Melissa MARTINETTI, Bridgewater, NJ (US); Ariel HASKEL, East Brunswick, NJ (US); Yun XU, Langhorne, PA (US); David SURIANO, Edison, NJ (US); Connie GREGSON, Princeton, NJ (US)

(73) Assignee: Colgate-Palmolive Company, New York, NY (US)

(21) Appl. No.: 17/766,979

(22) PCT Filed: Oct. 7, 2020
(86) PCT No.: PCT/US2020/054467

§ 371 Date: Apr. 6, 2022

Related U.S. Application Data
(60) Provisional application No. 62/911,649, filed on Oct. 7, 2019.

(51) Int. Cl. A61K 8/9789; A61Q 11/00

(52) U.S. Cl. CPC A61K 8/9789; A61Q 11/00; A61K 2800/48; A61K 2800/596

(57) ABSTRACT

This invention relates to oral care compositions comprising a cannabinoid source, a thickening system, wherein the thickening system comprises xanthan gum and carrageenan, and an orally acceptable carrier. The invention also relates to oral care compositions comprising a cannabinoid source, a surfactant system, wherein the surfactant system comprises an acyl glutamate, a non-silica abrasive (e.g., calcium carbonate or charcoal) and an orally acceptable carrier. Also disclosed are methods of using and of making these compositions.

This invention relates to oral care compositions comprising a cannabinoid source, a thickening system, wherein the thickening system comprises xanthan gum and carrageenan, and an orally acceptable carrier. Also disclosed are methods of using and of making these compositions. In another aspect the invention also relates to oral care compositions comprising a cannabinoid source, a surfactant system and a non-silica abrasive, wherein the surfactant system comprises and acyl glutamate, e.g., wherein the acyl glutamate may be C₈₋₂₅ acyl glutamate, e.g., C₈₋₁₈ acyl glutamate, or C₁₀₋₁₈ acyl glutamate, e.g., sodium cocoyl glutamate.

### BACKGROUND

Cannabinoids are a class of diverse chemical compounds that act on cannabinoid receptors in cells that alter neurotransmitter release in the brain. There are at least 113 different cannabinoids isolated from Cannabis, Cannabidiol (CBD) is one cannabinoid that has come into recent focus exhibiting varied effects. While delta-9-tetrahydrocannabinol (THC) is the major active ingredient of Cannabis extracts, cannabidiol makes up about 40% of Cannabis extracts and has been studied for many different uses. It is known that cannabidiol lacks the psychoactive effects seen in many of the other cannabinoids including delta-9-tetrahydrocannabinol (THC).

Unchecked bacterial growth in the oral cavity can lead to a number of adverse conditions. For example, gingivitis is an inflammation of the gums, and is one of the most common disorders of the oral cavity. It is ordinarily caused by bacterial accumulations on the surface of the teeth, which may be in the form of plaque. Gingivitis results in a number of unpleasant symptoms including inflamed gums that are painful or sensitive, halitosis, and bleeding from the gums while brushing or flossing. Other common disorders of the mouth include abscesses and cold sores, which also involve inflammation and are painful to those afflicted.

Accordingly, there is a need for oral care compositions with anti-bacterial efficacy, but which are also palatable and desirable for a user.

### BRIEF SUMMARY

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced. in their entireties. In the event of a conflict in a definition in the present disclosure and that of a cited reference, the present disclosure controls.

Without being bound by theory, it is believed that oral care compositions comprising cannabinoids such as CBD provide antibacterial and anti-inflammatory properties, for example, and are well-suited to treat a variety of oral diseases and disorders.

In one aspect, the present invention relates to novel oral care compositions that comprises cannabidiol (CBD). The structure of CBD is shown below:

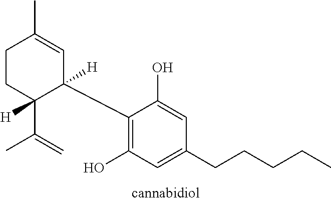 [SMILES: C=C(C)[C@@H]1CCC(C)=C[C@H]1c1c(O)cc(CCCCC)cc1O]

In one aspect the invention is an oral care composition (Composition 1.0)

comprising:
a. A cannabinoid source;
b. A thickening system, wherein the thickening system comprises xanthan gum and carrageenan; and
c. An orally acceptable vehicle.

For example, the invention contemplates any of the following compositions (unless otherwise indicated, values are given as percentage of the overall weight of the composition)

1.1 Composition of 1.0, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA), and combinations thereof.

1.2 Composition of 1.0 or 1.1, wherein the cannabinoid source comprises is a non-psychoactive cannabinoid.

1.3 Any of the preceding compositions, wherein the cannabinoid source comprises less than 0.3 wt. % Δ⁹-tetrahydrocannabinol (THC) relative to the total weight of the composition.

1.4 Any of the preceding compositions, wherein the cannabinoid source comprises less than 0.1 wt. % Δ⁹-tetrahydrocannabinol (THC) relative to the total weight of the composition.

1.5 Any of the preceding compositions, wherein the cannabinoid source comprises less than 0.01 wt. % Δ⁹-tetrahydrocannabinol (THC) relative to the total weight of the composition.

1.6 Any of the preceding compositions, wherein the cannabinoid source is substantially free of Δ⁹-tetrahydrocannabinol (THC).

1.7 Any of the preceding compositions, wherein the cannabinoid source comprises a cannabinoid selected from cannabichromene (CBC), cannabigerol (CBG), cannabidiol (CBD), and cannabinol (CBN), and combinations thereof.

1.8 Any of the preceding compositions, wherein the cannabinoid source comprises:

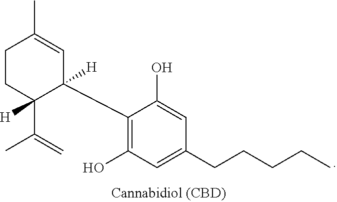 [SMILES: C=C(C)[C@@H]1CCC(C)=C[C@H]1c1c(O)cc(CCCCC)cc1O]

1.9 Any of the preceding compositions, wherein the cannabinoid source comprises hemp seed oil (HSO) or Cannabis sativa seed oil (CSO) or hemp oil (e.g., wherein the HSO or CSO or hemp oil are carriers for one or more cannabinoids).

1.10 The composition of 1,9, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA), and combinations thereof.

1.11 The composition of 1.10, wherein the cannabinoid source comprises cannabidiol (CBD).

1.12. Any of the preceding oral care compositions, wherein the cannabinoid source comprises one or more cannabinoids selected from: cannabichromene (CBC), cannabigerol (CBG), cannabidiol (CBD), and/or cannabinol (CBN), and wherein the one or more cannabinoids are present in an amount of 0.005 wt. % to 3.0 wt. 0.01 wt. % to 0.8 wt. %, 0.1% to 0.5%, 0.2 wt. % to 0.4 wt, %, 0.005 wt. %, 0.01 wt. %, 0.025 wt. %, 0.05 wt. %, 0.1% by wt. or 0.3 wt. % relative to the total weight of the composition.

1.13 Any of the preceding compositions, wherein the cannabinoid source comprises cannabidiol (CBD). (e.g., wherein the cannabinoid source comprises hemp oil and cannabidiol).

1.14 Any of the preceding compositions, comprising cannabidiol in an amount of 0.005 wt. % to 3.0 wt. %, 0.01 wt. % to 0.8 wt. %, 0.1% to 0.5%, 0,2 wt. % to 0.4 wt. %, about 0.005 wt. %, about 0.01 wt. %, about 0.025 wt. about 0.05 wt. %, about 0.1 wt. %, about 0.2 wt. %, or about 0.3 wt. % relative to the total weight of the composition.

1.15 Any of the preceding compositions, wherein the thickener system is present in an amount of 0.05 to 5% (e.g., 0.1%-2% by wt.) by weight relative to the total weight of the oral care composition.

1.16 Any of the preceding Compositions, wherein the xanthan gum is present at amount of, 0.1% to 0.5% (e.g., 0.1 to 5%) (e.g., about 0.45%) by weight relative to the total weight of the oral care composition.

1.17 Any of the preceding compositions, wherein the carrageenan is present in an amount of 0.1% to 0.5% (e.g., 0.1 to 2%) (e.g., about 0.20%) by weight relative to the total weight of the oral care composition.

1.18 Any of the preceding compositions, wherein the carrageenan is carrageenan concentrate.

1.19 Any of the preceding compositions, wherein the thickener system consists of xanthan gum and carrageenan.

1.20 Any of the preceding composition, wherein the wherein the ratio of xanthan gum to carrageenan is about 3:1 to about 1:1, (e.g., about 2.5:1 to about 1:1) (e.g., about 2.25:1).

1.21 Any of the preceding composition, wherein the composition further comprises an abrasive or particulate source.

1.22 The composition of 1.21, wherein the abrasive or particulate comprises one or more selected from sodium bicarbonate, calcium phosphate (e.g., dicalcium phosphate dihydrate), calcium sulfate, calcium carbonate (e.g., precipitated calcium carbonate) (e.g., natural calcium carbonate), calcium pyrophosphate, silica (e.g., hydrated silica) (e.g., precipitated silica), iron oxide, aluminum oxide, perlite, plastic particles, e.g., polyethylene, and combinations thereof.

1.23 Composition of 1.22, wherein the abrasive or particulate comprises one or more types of silica.

1.24 Composition of 1.23, wherein at least one type of silica is a synthetic abrasive silica. (e.g., 1%-25% by wt.) (e.g., 8%-25% by wt.) (e.g., about 12% by wt.)

1.25 Composition of 1.24, wherein at least one type of silica has an average particle size ranging from 2.5 microns to 12 microns.

1.26 Any of compositions 1.22-1.25, wherein at least one type of silica is a small particle silica having a median particle size (d50) of 1-5 microns (e.g., 3-4 microns) (e.g., about 5 wt. % Sorbosil AC43 from Ineos Silicas, Warrington, United Kingdom).

1.27 Any of the preceding compositions comprising 15-30 wt % of total silica in the composition.

1.28 Any of the preceding compositions, wherein at least one type of silica is small particle silica (e.g., having a median particle size (d50) of 3-4 microns) and wherein the small particle silica is about 10 wt. % of the oral care composition.

1.29 Any of the preceding compositions comprising silica wherein the silica is used as a thickening agent, e.g., particle silica.

1.30 Any of the preceding compositions, wherein the composition further comprises a zinc ion source.

1.31 A composition according to 1.30, wherein the zinc ion source comprises a zinc salt selected from the group consisting of: zinc citrate, zinc oxide, zinc phosphate, zinc lactate, zinc sulfate, zinc silicate, zinc gluconate and combinations thereof (e.g., zinc citrate and zinc oxide) (e.g., zinc phosphate) (e.g., zinc lactate),

1.32 Any of the preceding compositions, wherein the composition comprises a stannous ion source.

1.33 A composition according to 1.32, wherein the stannous ion source is selected from the group consisting of: stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide, or a mixture thereof.

1.34 A composition according to 1.33, wherein the stannous ion source is stannous fluoride.

1.35 Any of the preceding compositions, wherein the composition comprises a copolymer.

1.36 The composition of 1.35, wherein the copolymer is a PVM/MA copolymer.

1.37 The composition of 1.36, wherein the PVM/MA copolymer comprises a 1:4 to 4:1 copolymer of maleic anhydride or acid with a further polymerizable ethylenically unsaturated monomer; for example 1:4 to 4:1, e.g. about 1:1.

1.38 Any of the preceding compositions, wherein the further polymerizable ethylenically unsaturated monomer comprises methyl vinyl ether (methoxy ethylene).

1.39 Any of the preceding compositions, wherein the PVM/MA copolymer comprises a copolymer of methyl vinyl ether/maleic anhydride, wherein the anhydride is hydrolyzed following copolymerization to provide the corresponding acid.

1.40 Any of the preceding compositions, wherein the PVM/MA copolymer comprises a GANTREZ® polymer (e.g., GANTREZ® S-97 polymer)

1.41 Any of the preceding compositions wherein the pH is between 7.5 and 10.5. e.g., about 7.5 or about 8.0.

1.42 Any of the preceding compositions further comprising a fluoride ion source.

1.43 The composition of 1.42, wherein the fluoride ion source is selected from the group consisting of stannous fluoride, sodium fluoride, potassium fluoride, sodium monofluorophosphate, sodium fluorosilicate, ammonium fluorosilicate, amine fluoride, ammonium fluoride, and combinations thereof

1.44 The composition of 1.43, wherein the fluoride ion source is sodium fluoride and/or sodium monofluorophosphate.

1.45 Any of the preceding compositions wherein the polyphosphate is sodium tripolyphosphate (STPP).

1.46 The composition of 1.45, wherein the sodium tripolyphosphate is from 0.5-5.0 wt % (e.g., about 3.0 wt %).

1.47 Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, disodium hydrogenorthophosphate, monosodium phosphate, pentapotassium triphosphate and mixtures of any of two or more of these, e.g., in an amount of 1-20%, e.g., 2-8%, e.g., ca. 5%>, by weight of the composition.

1.48 Any of the preceding compositions, wherein the orally acceptable vehicle comprises one or more of water, a thickener, a buffer, a humectant, a surfactant, a sweetener, a pigment, a dye, an anti-caries agent, an anti-bacterial, a whitening agent, a desensitizing agent, a vitamin, a preservative, and mixtures thereof.

1.49 Any of the preceding compositions further comprising an anionic surfactant, wherein the anionic surfactant is in an amount of from 0.5 -5% by wt., e.g, 1-2% by weight, selected from water-soluble salts of higher fatty acid monoglyceride monosulfates, (e.g., sodium N-methyl N-cocoyl taurate), sodium cocomo-glyceride sulfate; higher alkyl sulfates, (e.g., sodium lauryl sulfate); higher alkyl-ether sulfates (e.g., of formula CH₃(CH₂)ₘCH₂(OCH₂CH₂)ₙOSO₃X, wherein m is 6-16, e.g., 10, n is 1-6, e.g., 2, 3 or 4, and X is Na) or (e.g., sodium laureth-2 sulfate (CH₃(CH₂)₁₀CH₂(OCH₂CH₂)₂OSO₃Na); higher alkyl aryl sulfonates (e.g., sodium dodecyl benzene sulfonate, sodium lauryl benzene sulfonate); higher alkyl sulfoacetates (e.g., sodium lauryl sulfoacetate; dodecyl sodium sulfoacetate), higher fatty acid esters of 1,2 dihydroxy propane sulfonate, sulfocolaurate (e.g., N-2-ethyl laurate potassium sulfoacetamide) and sodium lauryl sarcosinate, and mixtures thereof.

1.50 Any of the preceding compositions, wherein the anionic surfactant is sodium lauryl sulfate (e.g., from 1.0-2.0% by wt.) (e.g., from 1.0-1.5% by wt.) (e.g., about 1.2% by wt.).

1.51 Any of the preceding composition comprising a humectant.

1.52 The composition of 1.51, wherein the humectant comprises one or more selected from glycerin, sorbitol, xylitol, propylene glycol, polyols and combinations thereof.

1.53 A composition of 1.51, wherein the humectant comprises glycerin, wherein the glycerin is in a total amount of 5-20% by wt. (e.g., about 10% by wt.).

1.54 A composition of any of 1.51 1.53 comprising sorbitol (e.g., 10%-50% by wt.) (e.g., about 35.0% by wt.).

1.55 A composition of any of 1.51 1,54 comprising xylitol (e.g., in a total amount of 5-20% by wt.) (e.g., about 10% by wt.).

1.56 Any of the preceding compositions comprising polymer films.

1.57 Any of the preceding compositions comprising a flavoring agent, fragrance and/or coloring.

1.58 The composition of 1.57, wherein the flavoring agent is sodium saccharin, sucralose, or a mixture thereof.

1.59 Any of the preceding compositions comprising from 5% 40%, e.g., 10% 35%, e.g., about 10%, about 12%, about 14%, about 15%, about 18%, about 20%, about 25%, about 30%, and about 35% water.

1.60 Any of the preceding compositions comprising an additional antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, Zinc Chloride, Zinc Lactate, Zinc Sulfate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.

1.61 Any of the preceding compositions comprising an antioxidant, e.g., selected from the group consisting of Co-enzyme Q10, PQQ, Vitamin C, Vitamin E, Vitamin A, BHT, anethole-dithiothione, and mixtures thereof.

1.62 Any of the preceding compositions comprising a whitening agent.

1.63 The composition of 1,62, wherein the whitening agent is titanium dioxide.

1.64 Any of the preceding compositions comprising a whitening agent selected from a whitening active selected from the group consisting of peroxides, metal chlorites, perborates, percarbonates, peroxyacids, hypochlorites, and combinations thereof.

1.65 Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.

1.66 Any of the preceding compositions further comprising an agent that interferes with or prevents bacterial attachment, e.g., ELA or chitosan.

1.67 Any of the preceding compositions further a buffer system; (e.g. wherein the buffer comprises trisodium citrate and citric acid).

1.68 Any of the preceding compositions, wherein the composition comprises an aqueous buffer system, for example, wherein the buffer system comprises an organic acid and an alkali metal salt thereof, e.g., wherein the organic acid is citric acid and the salt is a mono-, di- and/or tri-alkali metal citrate salt, e.g., mono-, di- and/or tri-lithium, sodium, potassium, or cesium citrate salt, and citric acid. For example, where the composition comprises 1-10% by weight organic acid salt and 0.1-5% by weight organic acid.

1.69 Composition of 1.68, wherein the buffer system comprises a citrate buffer, wherein the citrate buffer comprises tri-sodium citrate and citric acid (e.g., 1 to 10% by weight of the composition), for example, wherein the molar ratio of mono-, di- and/or tri-sodium citrate and citric acid is 1.5 to 5, (e.g., 2 to 4).

1.70 Any of the preceding composition comprising a preservative (e.g., benzyl alcohol) (e.g., natural benzyl alcohol).

1.71 Any of the preceding compositions comprising:

a. A cannabinoid source comprising an oil (e.g., Cannabis sativa seed oil (CSO)) and cannabidiol, wherein the cannabinoid source comprises from 0.01%-0.05% by wt. of cannabidiol based on the total weight of the composition (e.g., about 0.025 by wt of cannabidiol, relative to the total weight of the composition) (e.g., where the oil is CSO used as a carrier for the cannabidiol).

b. a thickener system comprising xanthan gum and carrageenan (e.g., about 0.45% by wt. of xanthan gum) (e.g., about 0.20% by wt. of carrageenan) (e.g., carrageenan concentrate)

c. sorbitol (e.g., sorbitol 70% solution)

d. glycerin

e. a particulate source (e.g., silica)

f. benzyl alcohol (e.g., natural benzyl alcohol); and

g. an anionic surfactant (e.g., sodium lauryl sulfate)

1.72 Any of the preceding compositions comprising:

a. About 0.5% by wt of a cannabinoid source (e.g. a cannabinoid source comprising Cannabis sativa seed oil (CSO) and cannabidiol) (e.g., wherein the cannabinoid source comprises from 0.01%-0.05% by wt. of cannabidiol based on the total weight of the composition (e.g.., about 0.025 by wt of cannabidiol, relative to the total weight of the composition)

b. About 0.65% by wt of a thickener system comprising xanthan gum and carrageenan (e.g., about 0.45% by wt. of xanthan gum) (e.g., about 0.20% by wt. of carrageenan) (e.g., carrageenan concentrate)

c. About 35% by wt of sorbitol (e.g., sorbitol 70% solution)

d. About 10% by wt. of glycerin

e. About 20% by wt of a particulate source (e.g., silica)

1.73 Any of the preceding compositions effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit malodor, (vi) promote healing of sores or cuts in the mouth, (vii) reduce levels of acid producing bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health, e.g., by reducing potential for systemic infection via the oral tissues.

1.74 A composition obtained or obtainable by combining the ingredients as set forth in any of the preceding compositions.

1.75 Any the preceding oral care compositions, wherein the composition may be any of the following selected from the group consisting of: a toothpaste or a dentifrice, a mouthwash or a mouth rinse, a topical oral gel (e.g., an oral gel meant for office or professional use), a chewing gum, a dental tray application, mouth spray, foam, tablet, powder, a non-abrasive gel, a mousse, a denture cleanser, a coated or impregnated immediate or delayed release oral adhesive strip or patch, and a coated or impregnated oral wipe or swab.

1.76 Any of the preceding oral care compositions, wherein the composition is incorporated into a toothpaste.

1.77 Any of the preceding oral care compositions, wherein the cannabinoid source comprises substantially pure cannabidiol (CBD) (e.g., wherein the amount of CBD (by wt %) is 90%, 95%, or 99% or more (by wt %) of the total amount of cannabinoids in the oral care composition, by total wt% of the oral care composition).

1.78 Any of the preceding compositions, wherein the cannabinoid source comprises hemp oil and a cannabinoid (e.g., cannabidiol) and wherein the hemp oil is the hemp oil is blended with a medium chain triglyceride (MCT) oil or a hemp seed oil (e.g., wherein the hemp oil is a carrier for the cannabinoid).

A composition for use as set forth in any of the preceding compositions.

In another embodiment, the invention encompasses a method to improve oral health comprising applying an effective amount of the oral composition of any of the embodiments (e.g., any of Compositions 1.0 et seq) set forth above to the oral cavity of a subject in need thereof, e.g., a method to

i. reduce or inhibit formation of dental caries,

ii. reduce levels of acid producing bacteria,

iii. inhibit microbial bio film formation in the oral cavity,

iv. reduce plaque accumulation,

v. immunize (or protect) the teeth against cariogenic bacteria and their effects, and/or

vi. clean the teeth and oral cavity.

In still another aspect, the invention contemplates that any of Composition 1.0 et seq, can be used in a method to treat pain in the oral cavity and/or soothe an affected area of the oral cavity, wherein the method comprises applying an effective amount of the oral composition of any of the embodiments (e.g., any of Compositions 1.0 et seq) set forth above to the oral cavity of a subject in need thereof.

In yet another aspect, the invention contemplates that any of Composition 1.0 et seq, can be used in a method to reduce gingival irritation in the oral cavity, wherein the method comprises applying an effective amount of the oral composition of any of the embodiments (e.g., any of Compositions 1.0 et seq) set forth above to the oral cavity of a subject in need thereof.

In a further aspect, the invention contemplates that any of Composition 1.0 et seq, can be used in a method to promote a moisturizing effect in the oral cavity, wherein the method comprises applying an effective amount of the oral composition of any of the embodiments (e.g., any of Compositions 1.0 et seq) set forth above to the oral cavity of a subject in need thereof.

The invention further comprises the use of sodium bicarbonate, sodium methyl cocoyl taurate (tauranol), MIT, and benzyl alcohol and combinations thereof in the manufacture of a Composition of the Invention, e.g., for use in any of the indications set forth in the above method of Composition 1.0, et seq.

In another aspect the invention is an oral care composition (Composition 2.0) comprising:
a. a cannabinoid source;
b. a surfactant system, wherein the surfactant system comprises an acyl glutamate, e.g., wherein the acyl glutamate may be C₈₋₂₅ acyl glutamate, e.g., C₈₋₁₈ acyl glutamate, or C₁₀₋₁₈ acyl glutamate, (e.g., sodium cocoyl glutamate);
c. a non-silica abrasive (e.g., calcium carbonate or charcoal (e.g., activated charcoal)); and
d. an orally acceptable vehicle.

For example, the invention contemplates any of the following compositions (unless otherwise indicated, values are given as percentage of the overall weight of the composition)

2.1 Composition of 2.0, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CM), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA), and combinations thereof.

2.2 Composition of 2.0 or 2.1, wherein the cannabinoid source comprises is a non-psychoactive cannabinoid.

2.3 Any of the preceding compositions, wherein the cannabinoid source comprises less than 0.3 wt. % Δ⁹-tetrahydrocannabinol (THC) relative to the total weight of the composition.

2.4 Any of the preceding compositions, wherein the cannabinoid source comprises less than 0.1 wt. % Δ⁹-tetrahydrocannabinol (THC) relative to the total weight of the composition.

2.5 Any of the preceding compositions, wherein the cannabinoid source comprises less than 0.01 wt. % Δ⁹-tetrahydrocannabinol (THC) relative to the total weight of the composition.

2.6 Any of the preceding compositions, wherein the cannabinoid source is substantially free of Δ⁹-tetrahydrocannabinol (THC).

2.7 Any of the preceding compositions, wherein the cannabinoid source comprises a cannabinoid selected from cannabichromene (CBC), cannabigerol (CBG), cannabidiol (CBD), and cannabinol (CM), and combinations thereof.

2.8 Any of the preceding compositions, wherein the cannabinoid source comprises:

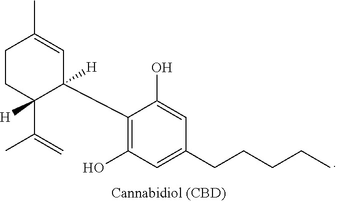 [SMILES: C=C(C)[C@@H]1CCC(C)=C[C@H]1c1c(O)cc(CCCCC)cc1O]

2.9 Any of the preceding compositions, wherein the cannabinoid source comprises hemp seed oil (HSO) or Cannabis sativa seed oil (CSO) or Hemp oil (e.g., wherein the HSO or CSO or Hemp oil are carriers for one or more cannabinoids).

2.10 The composition of 2.9, wherein the one or more cannabinoid is selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (URN), Δ⁹-tetrahydrocannabinol (THC), tetrahydrocannabinolic acid (THCA), and combinations thereof.

2.11 The composition of 2.10, wherein the cannabinoid source comprises cannabidiol (CBD).

2.12 Any of the preceding oral care compositions, wherein the cannabinoid source comprises one or more cannabinoids selected from: cannabichromene (CBC), cannabigerol (CBG), cannabidiol (CBD), and/or cannabinol (CBN), and wherein the one or more cannabinoids are present in an amount of 0.005 wt. % to 3.0 wt. %, 0.01 wt. % to 0.8 wt. %, 0.1% to 0.5%, 0.2 wt. % to 0.4 wt, %, 0.005 wt. %, 0.01 wt. %, about 0.025 wt. %, about 0.05 wt. %, about 0.1%, about 0.2%, or about 0.3 wt. % relative to the total weight of the composition.

2.13 Any of the preceding compositions, wherein the cannabinoid source comprises cannabidiol (CBD) (e.g., wherein the cannabinoid source comprises hemp oil and cannabidiol).

2.14 Any of the preceding compositions, comprising cannabidiol in an amount of 0.005 wt. % to 3.0 wt. %, 0.01 wt. % to 0.8 wt. %, 0.1% to 0.5%, 0.2 wt. % to 0.4 wt. %, about 0.005 wt. %, about 0.01 wt. about 0.025 wt. %, about 0.05 wt. %, about 0.1 wt. %, about 0.2 wt. %, or about 0.3 wt. % relative to the total weight of the composition.

2.15 Any of the preceding compositions, wherein the acyl glutamate is C₈₋₂₅ acyl glutamate, e.g., C₈₋₁₈ acyl glutamate, or C₁₀₋₁₈ acyl glutamate, optionally wherein the acyl glutamate is cocoyl glutamate, e.g., sodium cocoyl glutamate.

2.16 The composition of 2.15, wherein the acyl glutamate is cocoyl glutamate.

2.17 The composition of 2.16, wherein the cocoyl glutamate is sodium cocoyl glutamate.

2.18 Any of the preceding compositions, wherein the acyl glutamate (e.g., sodium cocoyl glutamate) is present in an amount of from 0.1% to 1%, e.g., from 0.3% to 0.6% by weight of the composition, e.g., about 0.5% by wt. of the composition.

2.19 Any of the preceding compositions wherein the non-silica abrasive comprises one or more selected from sodium bicarbonate, a calcium abrasive (e.g., calcium phosphate (e.g., dicalcium phosphate dihydrate), calcium sulfate, calcium carbonate (e.g., precipitated calcium carbonate) (e.g., natural calcium carbonate), or calcium pyrophosphate), iron oxide, aluminum oxide, perlite, plastic particles, e.g., polyethylene, and combinations thereof.

2.20 The composition of 2.16, wherein the calcium abrasive, e.g., calcium carbonate, is present in an amount of 1-10% by weight of the composition, e.g., 3-7% by weight of the composition, e.g., 4-6% by weight of the composition, e.g., about 5% by weight of the composition.

2.21 The composition of 2.19 or 2.20, wherein the calcium abrasive is calcium carbonate.

2.22 The composition of 2.2.1, wherein the calcium carbonate comprises natural calcium carbonate.

2.23 The composition of 2.22, wherein the natural calcium carbonate is present in an amount of 1-10% by weight of the composition, e.g., 3-7% by weight of the composition, e.g., 4-6% by weight of the composition, e.g., about 5% by weight of the composition.

2.24 The composition of 2.23, wherein the natural calcium carbonate is refined natural calcium carbonate.

2.25 The composition of 2,24, wherein the calcium carbonate comprises precipitated calcium carbonate.

2.26 The composition of 2.25, wherein the precipitated calcium carbonate is present in an amount of 1-10% by weight of the composition, e.g., 3-7% by weight of the composition, e.g., 4-6% by weight of the composition, e.g., about 5% by weight of the composition.

2.27 Any of the preceding compositions, wherein the calcium carbonate comprises natural calcium carbonate and precipitated calcium carbonate.

2.28 The composition of 2.27, wherein the natural calcium carbonate and precipitated calcium carbonate are present in an amount of 1-10% by weight of the composition, e.g., 3-7% by weight of the composition, e.g., 4-6% by weight of the composition, e.g., about 5% by weight of the composition.

2.29 Any of the preceding compositions, wherein the non-silica abrasive comprises charcoal (e.g., activated charcoal) (e.g., bamboo charcoal)

2.30 The composition of 2.29, wherein the charcoal is present in an amount of 1-10% by weight of the composition, e.g., 3-7% by weight of the composition, e.g., 4-6% by weight of the composition, e.g., about 5% by weight of the composition.

2.31 Any of the preceding compositions, wherein the composition further comprises a zinc ion source.

2.32 A composition according to 2.31, wherein the zinc ion source comprises a zinc salt selected from the group consisting of: zinc citrate, zinc oxide, zinc phosphate, zinc lactate, zinc sulfate, zinc silicate, zinc gluconate and combinations thereof (e.g., zinc citrate and zinc oxide) (e.g., zinc phosphate) (e.g., zinc lactate).

2.33 Any of the preceding compositions further comprising a fluoride ion source,

2.34 The composition of 2.33, wherein the fluoride ion source is selected from the group consisting of stannous fluoride, sodium fluoride, potassium fluoride, sodium monofluorophosphate, sodium fluorosilicate, ammonium fluorosilicate, amine fluoride, ammonium fluoride, and combinations thereof

2.35 The composition of 2.34, wherein the fluoride ion source is sodium fluoride and/or sodium monofluorophosphate.

2.36 Any of the preceding compositions wherein the polyphosphate is sodium tripolyphosphate (STPD).

2.37 The composition of 2.36, wherein the sodium tripolyphosphate is from 0.5-5.0 wt % (e.g., about 3.0 wt %).

2.38 Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, disodium hydrogenorthophosphate, monosodium phosphate, pentapotassium triphosphate and mixtures of any of two or more of these, e.g., in an amount of 1-20%, e.g., 2-8%, e.g., ca. 5%>, by weight of the composition.

2.39 Any of the preceding compositions, wherein the orally acceptable vehicle comprises one or more of water, a thickener, a buffer, a humectant, a surfactant, a sweetener, a pigment, a dye, an anti-caries agent, an anti-bacterial, a whitening agent, a desensitizing agent, a vitamin, a preservative, and mixtures thereof.

2.40 Any of the preceding compositions further comprising an anionic surfactant, wherein the anionic surfactant is in an amount of from 0.5 -5% by wt., e.g, 1-2% by weight, selected from water-soluble salts of higher fatty acid monoglyceride monosulfates, (e.g., sodium N-methyl N-cocoyl taurate), sodium cocomo-glyceride sulfate; higher alkyl sulfates, (e.g., sodium lauryl sulfate); higher alkyl-ether sulfates (e.g., of formula CH₃(CH₂)ₘCH₂(OCH₂CH₂)ₙOSO₃X, wherein m is 6-16, e.g., 10, n is 1-6, e.g., 2, 3 or 4, and X is Na) or (e.g., sodium laureth-2 sulfate (CH₃(CH₂)₁₀CH₂(OCH₂CH₂)₂OSO₃Na); higher alkyl aryl sulfonates (e.g., sodium dodecyl benzene sulfonate, sodium lauryl benzene sulfonate); higher alkyl sulfoacetates (e.g., sodium lauryl sulfoacetate; dodecyl sodium sulfoacetate), higher fatty acid esters of 1,2 dihydroxy propane sulfonate, sulfocolaurate (e.g., N-2-ethyl laurate potassium sulfoacetamide) and sodium lauryl sarcosinate, and mixtures thereof.

2.41 Any of the preceding compositions, wherein the anionic surfactant is sodium lauryl sulfate (e.g., from 1.0-2.0% by wt. (e.g., from 1.0-1.5% by wt.) (e.g., about 1.2% by wt.).

2.42 Any of the preceding composition comprising a humectant.

2.43 The composition of 2.42, wherein the humectant comprises one or more selected from glycerin, sorbitol, xanthan gum, xylitol, propylene glycol, polyols and combinations thereof

2.44 A composition of 2.43, wherein the humectant comprises glycerin, wherein the glycerin is in a total amount of 5-20% by wt. (e.g., about 10% by wt.).

2.45 A composition of any of 2.42-2.44 comprising sorbitol (e.g., 10% -50% by wt.) (e.g., about 35.0% by wt.).

2.46 A composition of any of 2.42 2.45 comprising xylitol (e.g., in a total amount of 5-20% by wt.) (e.g., about 10 % by wt.).

2.47 Any of the preceding compositions comprising polymer

2.48 Any of the preceding compositions comprising a flavoring agent, sweetener, fragrance and/or coloring.

2.49 The composition of 2.48, wherein the flavoring agent or sweetener is selected from: erythritol, Stevia rebaudiana leaf extract, sodium saccharin, sucralose, xylitol and mixtures thereof.

2.50 Any of the preceding compositions comprising from 5%-40%, e.g., 10%-35%, e.g., about 9%, about 8%, about 10%, about 12%, about 14%, about 15%, about 18%, about 20%, about 25%, about 30%, and about 35% water, by wt. of the total composition.

2.51 Any of the preceding compositions comprising an additional antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, Zinc Chloride, Zinc Lactate, Zinc Sulfate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, di octyl sulfosuccinate, salicylanilide, domiphen bromide, delmopinol, octapinol and other piperidino derivatives, ricin preparations, chlorite salts; and mixtures of any of the foregoing.

2.52 Any of the preceding compositions comprising an antioxidant, e.g., selected from the group consisting of Co-enzyme Q10, PQQ, Vitamin C, Vitamin E, Vitamin A, BHT, anethole-dithiothione, and mixtures thereof.

2.53 Any of the preceding compositions comprising a whitening agent.

2.54 The composition of 2.53, wherein the whitening agent is titanium dioxide.

2.55 Any of the preceding compositions comprising a whitening agent selected.

from a whitening active selected from the group consisting of peroxides, metal chlorites, perborates, percarbonates, peroxyacids, hypochlorites, and combinations thereof.

2.56 Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.

2.57 Any of the preceding compositions further comprising an agent that interferes with or prevents bacterial attachment, e.g., ELA or chitosan.

2.58 Any of the preceding compositions further a buffer system; (e.g. wherein the buffer comprises trisodium citrate and citric acid).

2.59 Any of the preceding compositions, wherein the composition comprises an aqueous buffer system, for example, wherein the buffer system comprises an organic acid and an alkali metal salt thereof, e.g., wherein the organic acid is citric acid and the salt is a mono-, di- and/or tri-alkali metal citrate salt, e.g., mono-, di- and/or tri-lithium, sodium, potassium, or cesium citrate salt, and citric acid. For example, where the composition comprises 1-10% by weight organic acid salt and 0.1-5% by weight organic acid.

2.60 Composition of 2.59, wherein the buffer system comprises a citrate buffer, wherein the citrate buffer comprises tri-sodium citrate and citric acid (e.g., 1 to 10% by weight of the composition), for example, wherein the molar ratio of mono-, di- and/or tri-sodium citrate and citric acid is 1.5 to 5, (e.g., 2 to 4).

2.61 Any of the preceding composition comprising a preservative (e.g., benzyl alcohol) (e.g., natural benzyl alcohol) (e.g., potassium sorbate).

2.62 Any of the preceding compositions further comprising one or more types of silica.

2.63 The composition of 2.62, wherein at least one type of silica is a precipitated silica. 1%-25% by wt. (e.g., 8%-25% by wt.) (e.g., about 12% by wt.)

2.64 The composition of 2.63, wherein at least one type of silica has an average particle size ranging from 2.5 microns to 12 microns.

2.65 Any of the preceding compositions comprising 15-30 wt % (e.g., about 20% by wt.) of total silica in the composition.

2.66 Any of the preceding compositions comprising silica wherein the silica is used as a thickening agent, e.g., particle silica.

2.67 Any of the preceding compositions comprising one or more natural ingredients selected from the group consisting of: Salix alba (white willow) bark, cocos nucifera (coconut) oil, and melaleuca alternifolia (tea tree) oil.

2.68 Any of the preceding compositions comprising:
a. A cannabinoid source comprising an oil (e.g., hemp oil) and cannabidiol, wherein the cannabinoid source comprises from 0.01%-0.5% by wt. of cannabidiol based on the total weight of the composition (e.g., about 0.1% by wt of cannabidiol, relative to the total weight of the composition) (e.g., where the oil is hemp oil used as a carrier for the cannabidiol))
b. a surfactant system, wherein the surfactant system comprises sodium cocoyl glutamate;
c. a non-silica abrasive comprising calcium carbonate; and
d. an orally acceptable vehicle.

2.69 Any of the preceding compositions comprising:
a. A cannabinoid source comprising hemp oil and cannabidiol, wherein the cannabinoid source comprises from 0.01%-0.5% by wt. of cannabidiol based on the total weight of the composition (e.g., about 0.1% by wt of cannabidiol, relative to the total weight of the composition) (e.g., where the hemp oil is a carrier for the cannabidiol))
b. a surfactant system, wherein the surfactant system comprises sodium cocoyl glutamate;
c. a non-silica abrasive comprising calcium carbonate;
d. an orally acceptable vehicle; and
e. wherein the composition does not contain an source of fluoride, 2.70 Any of the preceding compositions comprising:
a. A cannabinoid source comprising an oil (e.g., hemp oil) and cannabidiol, wherein the cannabinoid source comprises from 0.01%-0.5% by wt. of cannabidiol based on the total weight of the composition (e.g., about 0.1% by wt of cannabidiol, relative to the total weight of the composition) (e.g., where the oil is hemp oil which is used as a carrier for the cannabidiol))
b. a surfactant system, wherein the surfactant system comprises sodium cocoyl glutamate;
c. a non-silica abrasive comprising charcoal (e.g., activated charcoal); and
d. an orally acceptable vehicle.

2.71 Any of the preceding compositions effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit malodor, (vi) promote healing of sores or cuts in the mouth, (vii) reduce levels of acid producing bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity(xiv) reduce erosion, (xv) prevents stains and/or whiten teeth, (xvi) immunize the teeth against cariogenic bacteria; and/or (xvii) promote systemic health, including cardiovascular health, e.g., by reducing potential for systemic infection via the oral tissues.

2.72 A composition obtained or obtainable by combining the ingredients as set forth in any of the preceding compositions.

2.73 Any the preceding oral care compositions, wherein the composition may be any of the following selected from the group consisting of: a toothpaste or a dentifrice, a mouthwash or a mouth rinse, a topical oral gel (e.g., an oral gel meant for office or professional use), a chewing gum, a dental tray application, mouth spray, foam, tablet, powder, a non-abrasive gel, a mousse, a denture cleanser, a coated or impregnated immediate or delayed release oral adhesive strip or patch, and a coated or impregnated oral wipe or swab.

2.74 Any of the preceding oral care compositions, wherein the composition is incorporated into a toothpaste.

2.75 Any of the preceding oral care compositions, wherein the cannabinoid source comprises substantially pure cannabidiol (CBD) (e.g., wherein the amount of CBD (by wt %) is 90%, 95%, or 99% or more (by wt %) of the total amount of cannabinoids in the oral care composition, by total wt% of the oral care composition),

2.76 Any of the preceding oral care compositions, wherein the composition is free of any fluoride source.

2.77 Any of the preceding oral care compositions comprising an amphoteric surfactant (e.g., cocamidopropyl betaine) (e.g., a concentrated cocamidopropyl betaine).

2.78 The composition of 2.77, wherein the amount of the amphoteric surfactant (e.g., cocamidopropyl betaine) is from 0.1%-1% by wt. of the total composition (e.g., about 0.5% by wt).

2.79 Any of the preceding compositions, wherein the cannabinoid source comprises hemp oil and a cannabinoid (e.g., cannabidiol) and wherein the hemp oil is the hemp oil is blended with a medium chain triglyceride (MCT) oil or blended down with hemp seed oil (e.g., wherein the hemp oil is a carder for the cannabinoid).

A composition for use as set forth in any of the preceding compositions.

In another embodiment, the invention encompasses a method to improve oral health comprising applying an effective amount of the oral composition of any of the embodiments (e.g., any of Compositions 2.0 el seq) set forth above to the oral cavity of a subject in need thereof, e.g., a method to

vii. reduce or inhibit formation of dental caries,

viii, reduce levels of acid producing bacteria,

ix. inhibit microbial bio film formation in the oral cavity,

x. reduce plaque accumulation,

xi. immunize (or protect) the teeth against cariogenic bacteria and their effects, and/or

xii, clean the teeth and oral cavity.

In still another aspect, the invention contemplates that any of Composition 2.0 et seq, can be used in a method to treat pain in the oral cavity and/or soothe an affected area of the oral cavity, wherein the method comprises applying an effective amount of the oral composition of any of the embodiments (e.g., any of Compositions 1.0 et seq) set forth above to the oral cavity of a subject in need thereof.

In yet another aspect, the invention contemplates that any of Composition 2.0 et seq, can be used in a method to reduce gingival irritation in the oral cavity, wherein the method comprises applying an effective amount of the oral composition of any of the embodiments (e.g., any of Compositions 1.0 et seq) set forth above to the oral cavity of a subject in need thereof.

In a further aspect, the invention contemplates that any of Composition 2.0 et seq, can be used in a method to promote a moisturizing effect in the oral cavity, wherein the method comprises applying an effective amount of the oral composition of any of the embodiments (e.g., any of Compositions 2.0 et seq) set forth above to the oral cavity of a subject in need thereof.

The invention further comprises the use of sodium bicarbonate, sodium methyl cocoyl taurate (tauranol), MIT, and benzyl alcohol and combinations thereof in the manufacture of a Composition of the Invention, e.g., for use in any of the indications set forth in the above method of Composition 2.0, et seq.

### DETAILED DESCRIPTION

As used herein, the term “oral composition” means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, and the like.

As used herein, the term “dentifrice” means paste, gel, or liquid formulations unless otherwise specified. The dentifrice composition can he in any desired form such as deep striped, surface striped, multi-layered, having the gel surrounding the paste, or any combination thereof. Alternatively, the oral composition may be dual phase dispensed from a separated compartment dispenser.

### Cannabinoids

The term “cannabinoid” as used herein may refer to any compound that interacts with a cannabinoid receptor and other cannabinoid mimetics, including, but not limited. to, certain tetrahydropyran analogs (Δ⁹-tetrahydrocannabinol, Δ⁸-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1hydroxy-6,6-dimythel-9H-dibezo[b,d]pyran-9-ol, (−)-(3S,4S)-7-hydroxy-delta-6-tetrahydrocannabino1-1,1-dimethylheptyl, (+)-(3S,4S)-7-hydroxy-Δ-6-tetrahydrocannabinol, and Δ⁸-tetrahydrocannabinol-11-oic acid); certain piperidine analogs (e.g., (−)-(6S,6aR,9R,10aR)-5,6,6a,7,8,9,10,11.0a-octahydro-6-methyl-1-3-[(R′-1-methyl-4-phenylbutoxy]-1,9-phenanthridinediol 1-acetate)); certain aminoalkylindole analogs (e.g., (R)-(+)[2,3-dihydro-5-methyl-3-(4-morpholinylm-ethyl)-pyrrolo[1,2,3-de]-1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3′,4′,5′,6′-hexahydrobiphenyl), their salts, solvates, metabolites, and metabolic precursors.

The term “cannabidiol” as used herein refers to cannabidiol and cannabidiol derivatives. As used in this application, cannabidiol may be obtained from industrial hemp extract with a trace amount of THC (e.g., less than 0.3% by weight) or from Cannabis extract using high-CBD Cannabis cultivars.

The term “hemp seed oil” or “Cannabis sativa seed oil” refer to oil derived from hemp seed or Cannabis sativa seed. The term “hemp oil” refers to oil derived from Cannabis sativa (or hemp) flower, leaf, stem, or the whole plant, wherein the Cannabis sativa or hemp plant contains less than 0.3% by wt. THC. In one aspect, the hemp oil is blended with a medium chain triglyceride (MCT) oil. In another aspect, the hemp oil is blended with hemp seed oil.

In one aspect, Hemp oil, hemp seed oil, or Cannabis sativa seed oil can be incorporated in any of Composition 1.0 et seq or Composition 2.0 et seq, and can serve as delivery vehicle for the cannabinoid source. Cannabinoids utilized in the present invention, e.g., in any of Composition 1.0 et seq or Composition 2.0 et seq., may be in liquid form, as a natural (or additional) constituent of hemp oil, hemp seed oil or Cannabis sativa seed oil. “Hemp seed oil” (HSO) or “Cannabis sativa seed oil” (CSO) are used herein interchangeably. Hemp oil, HSO, or CSO, are harvested by cold pressing the seeds and the plants of the Cannabis sativa species. The resulting oil is extracted using CO₂ extraction or solvent extraction process, and may be further concentrated by distillation. Choice of cultivars may give different cannabinoid concentrations, but preferably, the targeted cannabinoids are cannabidiol (CBD) and cannabigerol (CBG). Other cannabinoids such as THC and cannabichromene (CBC) may also be present in hemp oil or Cannabis oil. Further isolation of these cannabinoids may result in solid, purified cannabinoids.

In certain embodiments, e.g., any of Composition 1.0 et seq or Composition 2.0 et seq, cannabinoids of the present invention may be present as isolates or extracts from the plants of the Cannabis sativa species.

In a preferred embodiment, e.g., any of Composition 1.0 et seq or Composition 2.0 et seq, toothpaste is manufactured with one or more cannabinoids incorporated for anti-bacterial effects. In this embodiment, the one or more cannabinoids are naturally derived or artificially derived.

When cannabinoids are provided in hemp oil or hemp seed oil Cannabis sativa seed oil, the hemp oil, hemp seed oil, or Cannabis sativa seed oil may contain up to 85% impurities, including fatty acids and other plant impurities. The extracted oil is then distilled to increase the cannabinoid concentration. Impurities in hemp oil, hemp seed oil and Cannabis sativa seed oil may be fatty acids such as linoleic acid and α-linoleic acid, which are natural components of hemp oil or Cannabis oil, β-caryophyllene, myrcene, and β-sitosterol.

In one aspect, cannabinoids may be provided e.g., in any of Composition 1.0 el seq . or Composition 2.0 et seq, as hemp oil, hemp seed oil or Cannabis sativa seed oil may contain impurities in an amount of less than 85%, less than 80%, less than 75%, less than 70%, less than 65?/r, less than 55%, less than 50%, less than 45%, less than 40%, or less than 35% by weight.

### Fluoride Ion Source

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No, 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., any of Composition 1.0 et seq or Composition 2.0 et seq) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The invention may in some embodiments contain anionic surfactants, e.g., the compositions of Composition 1.0, et seq. or Composition 2.0 et seq, for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 3 or 4, and X is Na or for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃₀ alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1 and optimal concentrations depend on the particular formulation and the particular surfactant. in one embodiment, the anionic surfactant is present at from 0.03% to 5% by weight, e.g., about 1.75% by wt.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of any of Composition 1.0, et seq. or Composition 2.0 et seq, that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain &alkyl sulfoxides and mixtures of such materials. In a particular embodiment, the composition of the invention comprises a nonionic surfactant selected from polaxamers (e.g., polaxamer 407), polysorbates (e.g., polysorbate 20) (e.g., polysorbate 80), polyoxyl hydrogenated castor oils (e.g., polyoxyl 40 hydrogenated castor oil), and mixtures thereof.

Illustrative amphoteric surfactants of Composition 1.0, et seq. or Composition 2.0 et seq, that can be used in the compositions of the invention include betaines (such as cocamidopropylbetaine) (e.g., concentrated cocamidopropylbetaine (e.g., tegobetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

In another aspect, the oral care composition of the invention (e.g., any of Composition 1.0 et seq or any of Composition 2.0 et seq) comprises a surfactant system comprising one or more of alkyl glucosides, acyl glutamates, glycolipids, or a combination thereof. As used herein, the terms “acyl glutamate” and “glycolipid” may be or include free form or any orally acceptable salts thereof. Alkyl glucosides which can be used in the composition may be C₈₋₂₅ alkyl glucoside, e.g., C₈₋₁₈ alkyl glucoside, or C₁₀₋₁₈ alkyl glucoside. The alkyl glucoside may be present in an amount of from 2% to 3%, e.g., from 2.2% to 2.8%, from 2.4% to 2.6%, or about 2.5% by weight of the composition. In some embodiments, the alkyl glucoside is lauryl glucoside. Acyl glutamates which can be used in the compositions may be C₈₋₂₅ acyl glutamate, e.g., C₈₋₁₈ acyl glutamate, or C₁₀₋₁₈ acyl glutamate. The acyl glutamate may be present in an amount of from 0.1%-5% by weight of the composition, e.g., about 0.1% by wt., about 0.2% by wt., about 0.3% by wt., about 0.4% by wt., or about 0.5% by wt. In some embodiments, the acyl glutamate is cocoyl glutamate, e.g., sodium cocoyl glutamate. The term “glycolipid” refers to any of a class of lipids that, upon hydrolysis, yield a sugar (e.g., galactose or glucose), and a lipid. The glycolipid may be present in an amount of from 0.1%-5% by weight of the composition, e.g., about 0.1% by wt., about 0.2% by wt., about 0.3% by wt., about 0.4% by wt., or about 0.5% by wt. In some embodiments, the glycolipid may be rhamnolipid, sophorolipid, trehalolipid, cellobiose lipid, or mannosylerythritol lipid. In some embodiments, the glycolipid is rhamnolipid, e.g., potassium rhamnolipid.

In some embodiments, the surfactant system comprises an alkyl glucoside and an acyl glutamate or the surfactant system comprises a glycolipid. In some embodiments, the surfactant system comprises a glucoside and an acyl glutamate. In some embodiments, the surfactant system comprises an acyl glutamate, e.g., cocoyl glutamate, e.g., sodium cocoyl glutamate, in an amount of from 0.1% 5% by weight of the composition, e.g., about 0.1% by wt,, about 0.2% by wt., about 0.3% by wt., about 0.4% by wt., or about 0.5% by wt. In some embodiments, the surfactant system comprises lauryl glucoside and cocoyl glutamate, e.g., sodium cocoyl glutamate.

The surfactant or mixtures of compatible surfactants can be present in the compositions of the present invention in 0.1% to 5%, in another embodiment 0.3% to 3% and in another embodiment 0,5% to 2% by weight of the total composition.

### Flavoring Agents

The oral care compositions of the invention may also include a flavoring agent. Flavoring agents which are used in the practice of the present invention, e.g, in any of Composition 1.0 et seq or Composition 2.0 et seq, include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 5% by weight.

### pH Adjusting Agents

In some embodiments, e.g., any of Composition 1.0 et seq or Composition 2.0 et seq, the compositions of the present disclosure contain a buffeting agent. Examples of buffering agents include anhydrous carbonates such as sodium carbonate, sesquicarbonates, bicarbonates such as sodium bicarbonate, silicates, bisulfates, phosphates (e.g., monopotassium phosphate, monosodium phosphate, disodium phosphate, dipotassium phosphate, tribasic sodium phosphate, sodium tripolyphosphate, pentapotassium tri polyphosphate, phosphoric acid), citrates (e.g. citric acid, trisodium citrate dehydrate), pyrophosphates (sodium and potassium salts, e.g., tetrapotassium pyrophosphate) and combinations thereof. The amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

### Chelating and Anti-Calculus Agents

The oral care compositions of the invention also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms, An effective amount of pyrophosphate salt useful in the present composition is generally enough to provide at least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt %. The pyrophosphates also contribute to preservation of the compositions by lowering water activity.

Suitable anticalculus agents for the invention (e.g., any of Composition 1.0 et seq or Composition 2.0 et seq) include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In particular embodiments, the invention includes alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g,, sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. in another embodiment, the compositions comprise a mixture of tetrasodium pyrophosphate (TSPP) and sodium tripolyphosphate (STPP)(Na₅P₃O₁₀), e.g., in proportions of TSPP at about 1-2% and STPP at about 7% to about 10%. Such phosphates are provided in an amount effective to reduce erosion of the enamel, to aid in cleaning the teeth, and/or to reduce tartar buildup on the teeth, for example in an amount of 2-20%, e.g., ca. 5-15%, by weight of the composition.

### Polymers

The oral care compositions of the invention also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

The N-vinyl-2-pyrrolidione is also commonly known as polyvinylpyrrolidone or “PVP”. PVP refers to a polymer containing vinylpyrrolidone (also referred to as N-vinylpyrrnlidone and N-vinyl-2-pyrrolidinone) as a monomeric unit. The monomeric unit consists of a polar imide group, four non-polar methylene groups and a non-polar methane group. The polymers include soluble and insoluble homopolymeric PVPs. Copolymers containing PVP include vinylpyrrolidone/vinyl acetate (also known as Copolyvidone, Copolyvidonum or VP-VAc) and vinyl pyrrolidone/dimethylamino-ethylmethacrylate. Soluble PVP polymers among those useful herein are known in the art, including Povidone, Polyvidone, Polyvidonum, poly(N-vinyl-2-pyrrolidinone), poly (N-vinylbutyrolactam), poly(1-vinyl-2-pyrrolidone) and poly [1-(2-oxo-1 pyrrolidinyl)ethylene]. These PVP polymers are not substantially cross-linked. In some embodiments the polymer comprises an insoluble cross-linked homopolymer. Such polymers include crosslinked PVP (often referred to as cPVP, polyvinylpolypyrrolidone, or cross-povidone).

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In some embodiments, microcrystalline cellulose (MCC) can be used (e.g., carboxymethyl cellulose with sodium carboxymethyl cellulose). An example of a source of MCC is Avicel® (FMC Corporation), which contains MCC in combination with sodium carboxymethyl cellulose (NaCMC). Both Avicel®. RC-591 (MCC containing 8.3 to 13.8 weight % NaCMC) and Avicel®. CL-611 (MCC containing 11.3 to 18.8 weight % NaCMC) may be used in certain aspects. In certain embodiments, the ratio of microcrystalline cellulose to cellulose ether thickening agent is from 1:1 to 1:3 by weight; or from 1:1.5 to 1:2.75 by weight. In any of the above embodiments comprising sodium carboxymethylcellulose, microcrystalline cellulose may be used in combination with NaCMC, In certain such embodiments, the MCC/sodium carboxymethylcellulose may be present in an amount of from 0.5 to 1.5 weight % based on the total weight of the composition.

### Abrasives

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 32.5 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica. In certain embodiments the invention may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used (e.g., Zeodent 165 or Zeodent 103 or Zeodent 113), such as precipitated silicas or silica gels. For example, synthetic amorphous silica. Silica may also be available as a thickening agent, e.g., particle silica. For example, the silica can also be small particle silica (e.g., Sorbosil AC43 from PQ Corporation, Warrington, United Kingdom). However, the additional abrasives are preferably not present in a type or amount so as to increase the RDA of the dentifrice to levels which could damage sensitive teeth, e.g., greater than 130.

### Water

Water is present in the oral compositions of the invention. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions, it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

The present invention in its method aspect involves applying to the oral cavity a safe and effective amount of the compositions described herein.

The compositions and methods according to the invention (e.g., any of Composition 1.0 et serif or Composition 2.0 et seq) can be incorporated into oral compositions for the care of the mouth and teeth such as dentifrices, toothpastes, transparent pastes, gels, mouth rinses, sprays and chewing gum.

As used throughout, ranges are used as shorthand for describing each and every , value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. it is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

Samples with varying amounts of cannabidiol (CBD) are assessed for their anti-oxidation ability. The samples include hemp seed oil (HSO) with varying concentrations of CBD, where the amount of CBD in the HSO of each sample is 5% by wt. relative to the weight of the HSO. The HSO can be considered a delivery vehicle for the CBD.

Samples containing 0.05% HSO and CBD (5% CBD by wt. of HSO), 0.1% HSO and CBD (5% CBD by wt. of HSO), 0.2% HSO and CBD (5% CBD by wt. of HSO), and 0.5% HSO and CBD (5% CBD by wt. of HSO) are tested in an assay to assess anti-oxidation performance. The amount (%) of CBD is relative to weight of the HSO. The samples are compared to untreated samples (negative control) as well as samples with vitamin E raw material (positive control).

Samples with 0.5% HSO and CBD (5% CBD by wt. of HSO) show comparable anti-oxidation capability compared to samples that contain vitamin E. Untreated samples are not believed to demonstrate any anti-oxidative efficacy, while samples with 0.05% HSO and CBD (5% CBD by wt. of HSO), 0.1% HSO and CBD (5% CBD by wt. of HSO), and 0.2% HSO and CBD (5% CBD by wt. of HSO) demonstrate increasing anti-oxidation efficacy, respectively, compared to the untreated samples.

Total Antioxidant Capacity Assay Kit (Abeam Catalog#: ab65329) is used to test raw material (full spectrum Hemp seed oil with 5% CBD) to assess anti-oxidation capacity of raw material. In this assay Cu2+ is used as proxy for the ROS (Reactive Oxygen Species)/Oxidized form. The transfer of an electron from an antioxidant molecule converts Cu2+ (oxidized form) to Cu+1(reduced form). Reduced Cu+ ion chelates with a colorimetric probe, giving a broad absorbance peak at 570 nm, which is proportional to the total antioxidant capacity. The kit gives antioxidant capacity in Trolox equivalents. Trolox, a water-soluble vitamin E analog, serves as an antioxidant standard.

Assay is conducted by using Cu2+ working solution (made by diluting 1 part of the Cu²⁺ reagent in 49 parts Assay Buffer). Place 100 μl of each sample and standard in a 96-well clear flat bottom plate. Add 100 μl of Cu²⁺ working solution to each well with samples or standards. After recommended incubation time plate is measured for absorbance at 570 nm. Data analysis is performed by creating a linear standard curve by plotting the concentration and absorbance of the standards. Standard curve is used to determine the concentration of the samples.

### Example 2

The assay described in Example 1 is conducted with toothpaste samples. The assay assesses a placebo toothpaste (i.e., which does not contain CBD), a CBD toothpaste, and vitamin E raw material. Similar to the results in Example 1, the CBI) toothpaste will demonstrate comparable anti-oxidation capability compared to the vitamin E raw material (positive control).

### Example 3

The following tables depict exemplary formulations of the present invention:

TABLE 1
Formulation A

| | (by wt %) |
|---|---|
| Water | q.s. (e.g., from 12%-15%) |
| Humectants | 45.0 |
| Abrasive Silica | 20.0 |
| Thickening Silica | 7.0 |
| Anionic Surfactant | 1.2 |
| Flavoring | 0.75-0.825 |
| Preservatives | 0.30-0.75 |
| Xanthan Gum | 0.45 |
| Carrageenan | 0.20 |
| Cannabis sativa seed oil | 0.5 |
| (5% CBD by wt. of the oil) | |
| Total Components | 100.0 |

TABLE 2
Formulation B

| | (by wt. %) |
|---|---|
| Water | q.s. (e.g., 10%-13%) |
| Humectants | 62.3 |
| Precipitated silica | 20 |
| Amphoteric Surfactant | 0.5 |
| Flavoring, color, sweeteners, | 1.75 |
| natural ingredients | |
| Sodium Cocoyl Glutamate | 0.5 |
| Sodium monofluorophosphate | 0.76 |
| Hemp Oil (30% CBD by wt. | 0.33 |
| of the oil) | |
| Calcium carbonate | 5 |
| Aloe vera | 0.05 |
| Total Components | 100.0 |

TABLE 3
Formulation C

| | (by wt. %) |
|---|---|
| Water | q.s. (e.g., 10%-13% by wt.) |
| Humectants | 62.3 |
| Precipitated silica | 20 |
| Amphoteric Surfactant | 0.5 |
| Flavoring, color, sweeteners, | 2.35 |
| natural ingredients | |
| Sodium Cocoyl Glutamate | 0.5 |
| Zinc citrate | 0.5 |
| Hemp Oil (30% CBD by wt. | 0.33 |
| of the oil) | |
| Calcium carbonate | 5 |
| Aloe vera gel | 0.05 |
| Preservative | 0.5 |
| Total Components | 100.0 |

TABLE 4
Formulation D

| | (by wt. %) |
|---|---|
| Water | q.s. (e.g., 7%-10%) |
| Humectants | 62 |
| Precipitated silica | 20 |
| Amphoteric Surfactant | 0.5 |
| Flavoring, color, sweeteners, | 2.2 |
| natural ingredients | |
| Sodium Cocoyl Glutamate | 0.5 |
| Hemp Oil (30% CBD by wt. | 0.33 |
| of the oil) | |
| Bamboo charcoal (3000 mesh) | 5 |
| Preservative | 0.5 |
| Total Components | 100.0 |

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

While the present invention has been described with reference to embodiments, it will be understood by those skilled in the art that various modifications and variations may be made therein without departing from the scope of he present invention as defined by the appended claims.

1. An oral care composition comprising:
a. A cannabinoid source;
b. A thickening system, wherein the thickening system comprises xanthan gum and carrageenan; and
c. An orally acceptable vehicle.

2. The composition of claim 1, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA), and combinations thereof.

3. (canceled)

4. (canceled)

5. The composition of claim 1, wherein the cannabinoid source comprises a cannabinoid selected from cannabichromene (CBC), cannabigerol (CBG), cannabidiol (CBD), and cannabinol (CBN), and combinations thereof.

6. The composition of claim 1, wherein the cannabinoid source comprises:

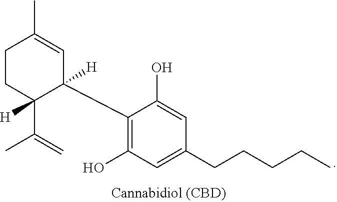 [SMILES: C=C(C)[C@@H]1CCC(C)=C[C@H]1c1c(O)cc(CCCCC)cc1O]

7. (canceled)

8. (canceled)

9. (canceled)

10. (canceled)

11. The composition according to claim 1, wherein the thickener system is present in an amount of 0.05 to 5% by weight relative to the total weight of the oral care composition.

12. The composition according to claim 1, wherein the xanthan gum is present at amount of, 0.1% to 0.5% by weight relative to the total weight of the oral care composition.

13. The composition of claim 1, wherein the carrageenan is present in an amount of 0.1% to 0.5% by weight relative to the total weight of the oral care composition.

14. (canceled)

15. (canceled)

16. The oral care composition of claim 1, wherein the oral composition may be any of the following oral compositions selected from the group consisting of: a toothpaste or a dentifrice, a mouthwash or a mouth rinse, a topical oral gel, a chewing gum, a dental tray application, mouth spray, foam, tablet, powder, a non-abrasive gel, a mousse, a denture cleanser, a coated or impregnated immediate or delayed release oral adhesive strip or patch, and a coated or impregnated oral wipe or swab.

17. (canceled)

18. A method to improve oral health comprising applying an effective amount of the oral composition of claim 1 to the oral cavity of a subject in need thereof.

19. A method to treat pain in the oral cavity and/or for soothing an affected area of the oral cavity, wherein the method comprises applying an effective amount of the oral composition of claim 1 to the oral cavity of a subject in need thereof.

20. An oral care composition comprising:
a. a cannabinoid source;
b. a surfactant system, wherein the surfactant system comprises an acyl glutamate;
c. a non-silica abrasive; and
d. an orally acceptable vehicle.

21. The composition of claim 20, wherein the cannabinoid source comprises a cannabinoid selected from cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ⁹-tetrahydrocannabinol (THC), tetrahydrocannabinolic acid (THCA), and combinations thereof.

22. (canceled)

23. (canceled)

24. The composition of claim 20, wherein the cannabinoid source comprises:

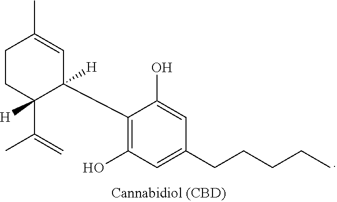 [SMILES: C=C(C)[C@@H]1CCC(C)=C[C@H]1c1c(O)cc(CCCCC)cc1O]

25. The composition of claim 24, wherein the cannabinoid source comprises hemp seed oil (HSO) or Cannabis sativa seed oil (CSO) or Hemp oil.

26. (canceled)

27. (canceled)

28. (canceled)

29. The composition of claim 20, wherein the non-silica abrasive is calcium carbonate.

30. The composition of claim 20, wherein the acyl glutamate is cocoyl glutamate.

31. The composition of claim 30, wherein the cocoyl glutamate is sodium cocoyl glutamate.

32. The composition of claim 20 comprising:
a. A cannabinoid source comprising hemp oil and cannabidiol, wherein the cannabinoid source comprises from 0.01%-0.5% by wt. of cannabidiol based on the total weight of the composition.
b. a surfactant system, wherein the surfactant system comprises sodium cocoyl glutamate;
c. a non-silica abrasive comprising calcium carbonate; and
d. an orally acceptable vehicle.

33. The composition of claim 20 comprising:
a. A cannabinoid source comprising hemp oil and cannabidiol, wherein the cannabinoid source comprises from 0.01%-0.5% by wt. of cannabidiol based on the total weight of the composition
b. a surfactant system, wherein the surfactant system comprises sodium cocoyl glutamate;
c. a non-silica abrasive comprising calcium carbonate;
d. an orally acceptable vehicle; and
e. wherein the composition does not contain a source of fluoride.

34. The composition of claim 20 comprising:
a. A cannabinoid source comprising hemp oil and cannabidiol, wherein the cannabinoid source comprises from 0.01%-0.5% by wt. of cannabidiol based on the total weight of the composition.
b. a surfactant system, wherein the surfactant system comprises sodium cocoyl glutamate;
c. a non-silica abrasive comprising charcoal; and
d. an orally acceptable vehicle.
